(19) United States
(12) Patent Application Publication
(10) Pub. No.: US 2025/0009288 A1
(43) Pub. Date: Jan. 9, 2025

(54) MONITOR SYSTEM AND METHOD FOR FETAL MOVEMENT ASSESSMENT

(71) Applicant: HERA MED LTD., Netanya (IL)

(72) Inventors: David GROBERMAN, Even Yehuda (IL); Michael NENNER, Tel Aviv (IL); Ariel LADEN, Pardes-Hanna-Karkur (IL); Arturo WESCHLER, Kfar Saba (IL); Maya STEELE, Givatayim (IL)

(21) Appl. No.: 18/711,079

(22) PCT Filed: Nov. 16, 2022
(86) PCT No.: PCT/IL2022/051220

§ 371 Date: May 16, 2024

Related U.S. Application Data
(60) Provisional application No. 63/280,772, filed on Nov. 18, 2021.

(51) Int. Cl. A61B 5/00

(52) U.S. Cl. CPC A61B 5/4362; A61B 5/7435; A61B 5/7445

(57) ABSTRACT

Monitoring and assessment of fetal movement with an input/output device (such as a touch screen) adapted to display a schematic representation of a pregnant woman's abdomen; at least one processor; a memory device; and at least one program adapted to receive input on the input/output device and record a fetal movement at a position in said pregnant woman's abdomen corresponding to a position of the input on the input/output device, and to count and save in the memory device a plurality of positions of fetal movements in said pregnant woman's abdomen over a recorded time period and to update the display with the position of the fetal movements in said recorded time period.

### FIELD OF THE INVENTION

The present invention generally relates to pregnancy monitoring, and in particular relates to the assessment of fetal development by pregnant users.

### BACKGROUND OF THE INVENTION

Maternal awareness of fetal movements is the simplest and oldest method of evaluating fetal wellbeing with the primary goal of stillbirth risk reduction. Fetal movement counting and other associated assessment was introduced into routine prenatal care more than 40 years ago, following the discovery of the association between decreased fetal movements and increased risk for stillbirth in the early 1970s. However, despite decades of use, no single standard tool or methodology has been adopted, and numerous different methods are in use. Methods for fetal movement assessment can be classified into formal (methods based on formal counting and other assessment of fetal movements for a predefined time or until a predefined number is achieved) and informal (methods based on subjective estimation of daily fetal movements without formal counting).

One of the main challenges in implementing formal fetal movement assessment is the low adherence to the different assessment protocols that provide fetal movement information due to their time-consuming character. Informal approaches that do not include structured movement counting have been used to identify reduced fetal movements, especially in multigravida women with experience of one or several previous pregnancies Due to a lack of tools in the prior art available to expectant mothers to facilitate structured movement assessment, the benefits of this general class of assessment to multigravida and primigravida expectant mothers remains unavailable. An objective of the present invention is thus to provide a structured fetal movement assessment system and method that can be easily implemented and reliably adhered to by expectant mothers, and which can store, communicate, and evaluate the data collected.

### SUMMARY OF THE INVENTION

The following embodiments and aspects thereof are described and illustrated in conjunction with systems, devices and methods which are meant to be exemplary and illustrative and not limiting in scope. In various embodiments, one or more of the above-described problems or drawbacks have been reduced or eliminated, while other embodiments are directed to other advantages or improvements.

According to an aspect of the invention a system for monitoring and assessment of fetal movement, comprising: an input/output device adapted to display a schematic representation of a pregnant woman's abdomen; at least one processor; a memory device; and at least one program adapted to receive input on the input/output device and record a fetal movement at a position in said pregnant woman's abdomen corresponding to a position of the input on the input/output device, and to count and save in the memory device a plurality of positions of fetal movements in said pregnant woman's abdomen over a recorded time period and to update the display with the position of the fetal movements in said recorded time period.

In another aspect of the invention, the input/output device is a touch screen.

In another aspect of the invention, the schematic representation of the pregnant woman's abdomen comprises a vertical y-axis, a curved, upwardly inflected x-axis generally perpendicular to the y-axis and crossing the y-axis at an origin representing a navel on the pregnant woman's abdomen, the axes thereby schematically dividing the pregnant women's abdomen into four quadrants.

In another aspect of the invention, the at least one processor is adapted to receive inputs from the input/output device representing a duration of said fetal movement and to record and save in the memory device a plurality of durations of fetal movements corresponding with the positions of fetal movements over recorded time period.

In another aspect of the invention, the processor is adapted to receive inputs from the input/output device representing an intensity of said fetal movement and to record and save in the memory device a plurality of intensity ratings of fetal movements corresponding with the positions of fetal movements over a recorded time period.

In another aspect of the invention, the input/output device and the at least one processor are components of a smart phone.

In another aspect of the invention, a means of wired or wireless communication or combination thereof is configured to transmit information relating to the fetal movement over a recorded time period to or from the processor.

In another aspect of the invention, the one or more programs is configured to present qualitative questions and input answer fields thereto to a pregnant user of the device.

In another aspect of the invention, a method may also be employed for the monitoring and assessment of fetal movement, comprising the following steps taken in any order: (a) displaying a schematic representation of a pregnant woman's abdomen on an input/output device, at least one processor, a memory device, and at least one program adapted to receive input on the input/output device; (b) receiving input on the input/output device; (c) recording a fetal movement at a position in said pregnant woman's abdomen corresponding to a position of the input on the input/output device; (d) counting a plurality of positions of fetal movements in said pregnant woman's abdomen over a recorded time period; and (e) saving the plurality of positions counted.

In another aspect of the invention, the schematic representation of the pregnant woman's abdomen employed in the method for monitoring and assessment of fetal movement is an image on the input/output device of a vertical y-axis and a curved upwardly inflected x-axis generally perpendicular to the y-axis and crossing the y-axis at an origin representing a navel on the pregnant woman's abdomen, the axes thereby schematically dividing the pregnant women's abdomen into four quadrants.

In another aspect of the invention, the at least one processor employed in the method for monitoring and assessment of fetal movement receives inputs from the input/output device representing the duration of fetal movements and records a plurality of durations of fetal movements corresponding with the positions of fetal movements over a recorded time period.

In other another aspect of the invention, the at least one processor employed in the method for monitoring and assessment of fetal movement receives inputs from the input/output device representing an intensity of said fetal movement; and records and saves in the memory device a plurality of intensity ratings of fetal movements corresponding with the positions of fetal movements recorded over the recorded time period.

In another aspect of the invention, a means of wired or wireless communication or a combination thereof may be employed in the method for monitoring and assessment of fetal movement to communicate information related to the fetal movement over a recorded time period to or from a remote computing device.

In another aspect of the invention, the method for monitoring and assessment of fetal movement comprises the additional steps of (f) presenting qualitative questions and associated answer input fields thereto to a pregnant user of the device; and (g) receiving input provided in input fields from the pregnant user of the device associating and recording the input from said fields information relating to said recorded time period.

In another aspect of the invention, the fetal movements recorded in the method for monitoring and assessment of fetal movements are recorded over discrete time periods and fetal movements recorded in different discrete time periods are compared.

In another aspect of the invention, the at least one processor employed in the method for the monitoring and assessment of fetal movements evaluates the differences between fetal movements recorded in the same time period or differences between fetal movements recorded in different time periods or a combination thereof.

In another aspect of the invention, a computing device for monitoring and assessment of fetal movement contains a non-transitory computer readable medium containing instructions that when executed cause the at least one processor to execute a method of monitoring and assessment of fetal movement.

### BRIEF DESCRIPTION OF THE DRAWINGS

Some embodiments of the invention are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the invention.

FIG. 1 is an illustration of the display of an input/output device having a schematic representation of a pregnant woman's abdomen according to an embodiment of the invention;

FIG. 2 is an illustration of the display of the input/output device of FIG. 1 mid-session after recording a single fetal movement, according to an embodiment of the invention;

FIG. 3 is an illustration of the display of the input/output device of FIG. 2 post-session according to n embodiments of the invention.

FIG. 4 is an illustration of a qualitative question and answer fields thereto, according to an embodiment of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

The term “session” as used herein relates the period of time between the initiation of the recording of specific fetal movements by the pregnant user of the device and the conclusion of the recording of said fetal movement.

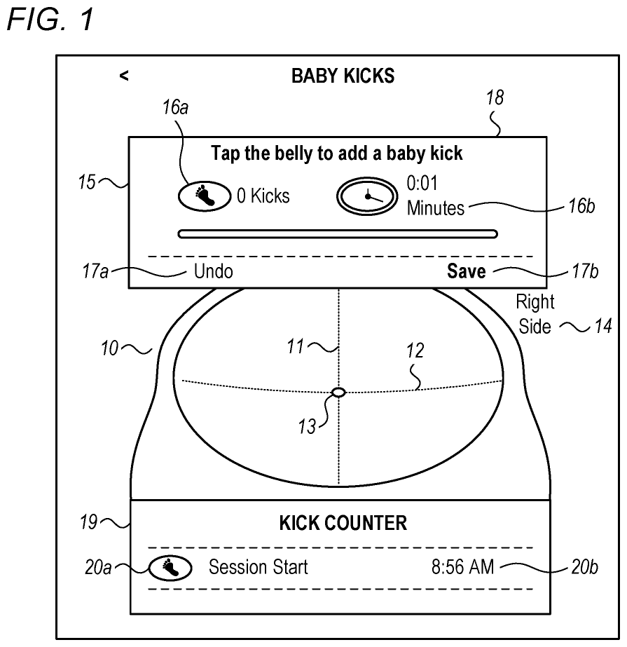

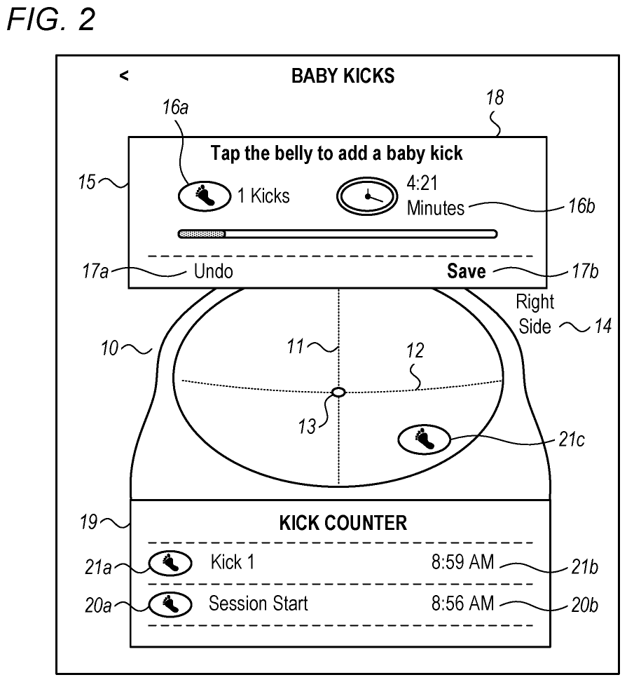

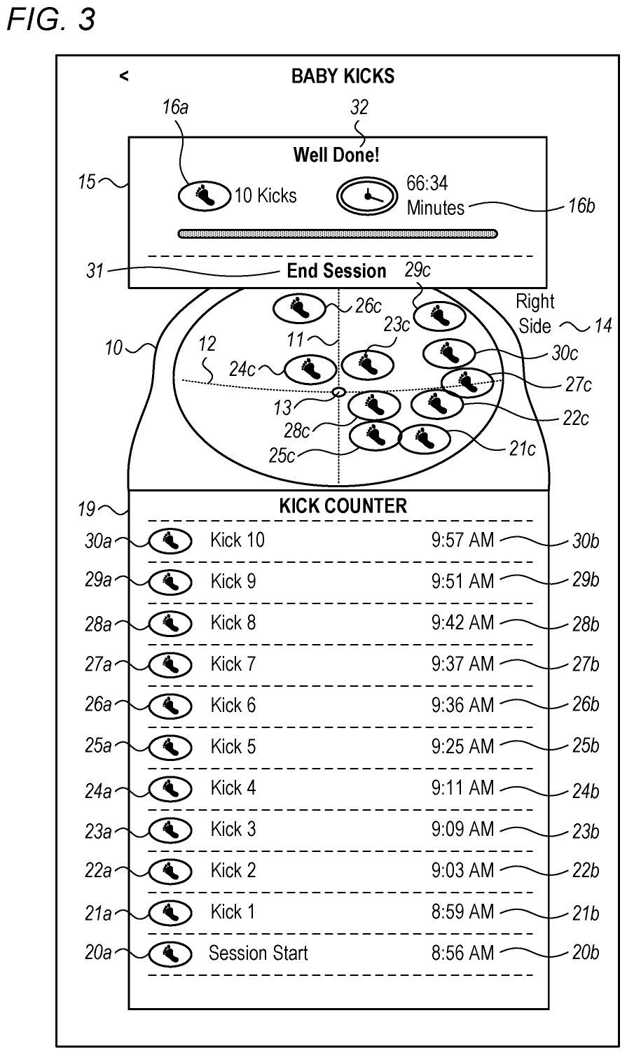

The term “kick” as used herein can relate to fetal movements in general or to a specific type of fetal movement wherein a pregnant user of the device subjectively defines the fetal movement as relating to the fast movement of a fetus's foot in their abdomen. The former, general, use of the term “kick” relates to instances where quick user-friendly information is required, such as in the use term “kick” in the display of the input/output device of a computing device of the number of fetal movements 16a or the kick counter 19, as presented in FIG. 1, in FIG. 2, and in FIG. 3.

According to some embodiments, the computing device and method for assessment and monitoring of fetal movements may be implemented on a smart phone comprising an input/output device. According to some embodiments, the computing device and method for assessment and monitoring of fetal movements may be implemented on a tablet device comprising an input/output device. According to some embodiments, the computing device and method for assessment and monitoring of fetal movements may be implemented on a laptop device containing an input/output device.

Reference is made to FIG. 1 which is an illustration of the display of the input/output device having a schematic representation 10 of a pregnant woman's abdomen, according to an embodiment of the invention. In the embodiment shown, the pregnant woman's abdomen is schematically divided into quadrants by a vertical y-axis 11 and a curved, upwardly inflected x axis 12 which crosses the y-axis, subjectively identified as the navel 13 of the pregnant woman. According to some embodiments, possible confusion arising from the perspective of the pregnant user of the device with respect to the spatial orientation of the schematic representation 10 in relation to a pregnant user's abdomen is mitigated with an orientation guide 14 indicating the orientation of the schematic representation 10 in relation to the pregnant user's abdomen. According to some embodiments, the display of the input/output device of the computing device presents a session interface 15 to the pregnant user, which presents: the number of fetal movements 16a; the time elapsed 16b; an option to undo an input 17a; an option to save 17b; and succinct instructions for use 18. According to some embodiments, the display of the input/output device of the computing device presents a kick counter 19 on which a list of events in a use session are recorded chronologically with corresponding event time information, including the start of the session 20a with corresponding event time information 20b.

According to some embodiments, pregnant users of the device may select a schematic representation 10 of a pregnant woman's abdomen in either a mirrored or non-mirrored configuration in relation to their perspective using the computing device. In the case of a pregnant user selecting a schematic representation of a pregnant woman's abdomen in a non-mirrored configuration, the orientation guide 14 would read “Left Side” and be located in the position of 14 in the case illustrated by FIG. 1 in some embodiments, or in other embodiments would read “Right Side” and be located on the opposite lateral side to the position of 14 in the case illustrated by FIG. 1.

Reference is made to FIG. 2, which is an illustration of the display of the input/output device mid-session of FIG. 1 after recording a single fetal movement, according to an embodiment of the invention. In the case illustrated by FIG. 2, a pregnant user has recorded a single fetal movement 21a, having time 21b and location 21c presented on the schematic representation 10. According to some embodiments, the pregnant user of the device has inserted the information relating to the time 21b and location 21c of the fetal movement 21a by inputting the input/output device of the computing device. According to some embodiments, the information relating to the fetal movement 21a also includes but is not limited to: the duration of the fetal movement; the type of the fetal movement; the intensity of the fetal movement, information which can be inserted by the pregnant user of the device after contacting by touch the touch display of the of the computing device. According to some embodiments, the number of fetal movements 16a displays a number with respect to the presence of fetal movement 21a, and the time elapsed 16b displays the time period of the session.

According to some embodiments, the duration of the pregnant user's touch on the input/output device of the computing device can be configured to relate to information relating to the duration of the fetal movement or information relating to the intensity of the fetal movement. According to some embodiments, when the computing devices comprises an input/output device capable of sensing pressure of touch contact, the pressure of the touch contact by the pregnant user on the input/output device relates to the intensity of the fetal movement.

Maternal perception of fetal movements as a measure of fetal wellbeing can include not only assessment of the number of movements but also perceived movement type and intensity and the diurnal variation between fetal movements or groupings thereof. In the case illustrated by FIG. 2, a location 21c and other qualities of a fetal movement perceived by the pregnant woman may be recorded. In the case illustrated by FIG. 2, the fetal movement 21a recorded at time 21b and location 21c is a “Kick” type, as illustrated by the baby foot icon, however the pregnant user of the device is able to select the type from a list of options including but not limited to: “Push”; “Pull”; “Hand”; “Leg”; “Back”; and “Kick”, each with their own illustrative icon, according to some embodiments. According to some embodiments, the default type setting for recorded fetal movements is “Kick”.

Reference is made to FIG. 3, which is an illustration of the display of the input/output device of FIG. 2 post-session according to an embodiment of the invention. In the case illustrated by FIG. 3, the user has selected the save option, illustrated as 17b in FIG. 1 and FIG. 2, thus concluding the session. The number of fetal movements 16a shows the number of fetal movements recorded during the session, and the time elapsed 16b shows the concluded period of the session. The instruction 18 has been replaced with a concluding message 32, according to some embodiments. In the case illustrated by FIG. 3, fetal movements 21a, 22a, 23a, 24a, 25a, 26a, 27a, 28a, 29a, 30a, are recorded in the kick counter 19, having recorded times 21b, 22b, 23b, 24b, 25b, 26b, 27b, 28b, 29b, 30b, respectively, and locations on the schematic representation 10 displayed 21c, 22c, 23c, 24c, 25c, 26c, 27b, 28b, 29b, 30b, respectively. In the case illustrated by FIG. 3, all fetal movements recorded are displayed as being of the “Kick” type.

According to some embodiments of the invention, the method and system for fetal movement monitoring and assessment includes a combination of qualitative data with quantitative data.

According to some embodiments, the quantitative portion of the method for fetal movement monitoring and assessment includes collecting a rich database of fetal movement characteristics including but not limited to: time; movement type; position on abdomen; intensity combined with user-specific data including but not limited to: gestational age of fetus; fetal positioning in utero; and number of previous pregnancies. This database can facilitate the realization of the predictive value of fetal movement data, such as by the application of machine learning methods to the movement database and to the documented clinical outcomes, according to some embodiments.

According to some embodiments, the database is stored and updated on the memory device of the computing device. According to some embodiments, the database is stored and updated on a remote device, wherein the computing device is in communication with the remote device via a wired means of communication or a wireless means of communication or a combination thereof. According to some embodiments, the database is stored and updated on both a memory device on a computing device and on a remote device, allowing for back-up functionality in the event of a failure of: the memory device; the computing device; or the remote device.

According to some embodiments, the fetal movement data in a database is correlated over days and times of pregnancy, which provides a broad database of the fetal movement, which can be then analyzed by a digital method or a healthcare worker or combination thereof to determine a pattern and thus to inform more knowledgeable medical decisions than would otherwise be available. According to some embodiments, a digital method for analyzing fetal movement data is integrated autonomously by the at least one processor on the computing device. According to some embodiments, data is presented in clear and comprehensive manner by the at least one processor on the computing device to a healthcare worker to facilitate efficient communication of data to inform the more knowledgeable medical decisions.

According to some embodiments, a pattern personalized to an individual pregnant user of the device can be determined to calculate a trend with averages and thresholds that may be calculated to analyze deviations from a baseline pattern which indicate fetal wellbeing and thus indicating potential distress or complication. This method can be conducted autonomously on the computing device and displayed to the pregnant user of the device, according to some embodiments.

According to some embodiments, the calculated trend and baseline patterns can further include “grouping” of movements, for example if a pregnant user of device records multiple instances of a between 5 and 15 fetal movements within 10 minutes followed by a period with no fetal movements.

According to some embodiments, a detailed “map” of fetal movements throughout the pregnant user's pregnancy may be produced by a method executed by the at least one processor using a fetal movement database in order to estimate the fetus's position, providing crucial information to the pregnant user and medical workers, specifically when the pregnancy is approaching its natural conclusion. Systems and methods disclosed in the prior art limit expectant mother and medical workers to utilizing ultrasound (US) imaging, the availability of which is unreliable, unaffordable, or simply impossible in many births, such as births in rural and remote areas and home births.

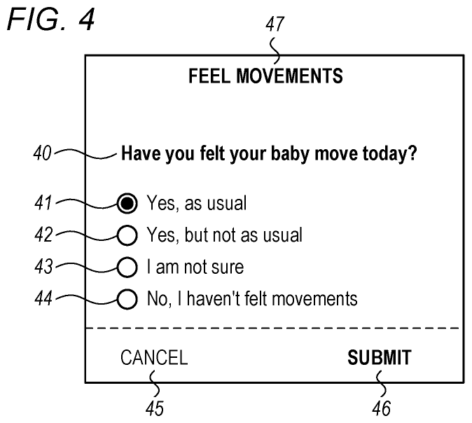

Reference is made to FIG. 4, which is an illustration of a qualitative question and answer fields thereto, according to an embodiment of the invention. On the input/output device of the computing device, a question 40 has four different possible input answer fields 41, 42, 43, and 44, according to some embodiments. In the case illustrated by FIG. 4, the input answer fields are mutually exclusive, and the pregnant user of the device has selected the first field 41 as their subjective answer to the question 40. According to some embodiments, the input/output device displays: an option to cancel the question 45; an option to submit the question with the input answer field selected 46; and a title of the question 47.

According to some embodiments, other information not directly related to a specific fetal movement may be collected by use of question and input answer fields thereto by use of input on the input/output device of the computing device. According to some embodiments, a simple qualitative assessment may be implemented by use of an input on the input/output device of a computing device such as depicted in FIG. 4, and may be used for daily screening, thus ensuring a satisfactory adherence level. According to some embodiments, the quantitative assessment is used only following a qualitative assessment determined to be of unreliable accuracy by the pregnant user of the device or the autonomous analytical method executed by the at least one processor, or when any concern is raised.

According to some embodiments, data collected with a computing device and a method of assessment and monitoring of fetal movement may be processed to predict a baby's health after birth, which may present throughout childhood, including chronic behavior such as attention deficit hyperactivity disorder or other clinical disorders involving excessive or decreased movement.

According to some embodiments, the recording of sessions permits comparison of data collected by a pregnant user of the device at different times of a day.

According to some embodiments, a bio-feedback system enables associating fetal movements with diverse external influence factors such as maternal stress level, specific sounds (such mother talking, music, etc.) in real time, and to correlating results with physical activity, nutrition (correlating the movement to specific diet plans).

Although the present invention has been described with reference to specific embodiments, this description is not meant to be construed in a limited sense. Various modifications of the disclosed embodiments, as well as alternative embodiments of the invention will become apparent to persons skilled in the art upon reference to the description of the invention. It is, therefore, contemplated that the appended claims will cover such modifications that fall within the scope of the invention.

What is claimed is:

1. A system for monitoring and assessment of fetal movement, comprising:
i) an input/output device adapted to display a schematic representation of a pregnant woman's abdomen;
ii) at least one processor;
iii) a memory device; and
iv) at least one program adapted to receive input from an input/output device and record a fetal movement at a position in said pregnant woman's abdomen corresponding to a position of the input on the input device, and to count and save in the memory device a plurality of positions of fetal movements in said pregnant woman's abdomen over a recorded time period and to update the display with the position of the fetal movements in said recorded time period.

2. The system of claim 1, wherein the input/output device is a touch screen.

3. The system of claim 1, wherein the schematic representation of the pregnant woman's abdomen comprises a vertical y-axis, a curved, upwardly inflected x-axis generally perpendicular to the y-axis and crossing the y-axis at an origin representing a navel on the pregnant woman's abdomen, the axes thereby schematically dividing the pregnant women's abdomen into four quadrants.

4. The system of claim 1, wherein the at least one processor is adapted to receive inputs from the input device representing a duration of said fetal movement and to record and save in the memory device a plurality of durations of fetal movements corresponding with the positions of fetal movements over a recorded time period.

5. The system of claim 1, wherein the processor is adapted to receive inputs from the input/output device representing an intensity of said fetal movement and to record and save in the memory device a plurality of intensity ratings of fetal movements corresponding with the positions of fetal movements over a recorded time period.

6. The system of claim 1, wherein the input/output device and the at least one processor are components of a smart phone.

7. The system of claim 1 wherein a means of wired or wireless communication or combination thereof is configured to transmit information relating to the fetal movement over a recorded time period to or from the processor.

8. The system of claim 1, wherein the at least one program is configured to present qualitative questions and input answer fields thereto to a user of the device.

9. A method for monitoring and assessment of fetal movement, comprising the following steps taken in any order:
a) displaying a schematic representation of a pregnant woman's abdomen on a input/output device;
b) receiving input from an input/output device;
c) recording a fetal movement at a position in said pregnant woman's abdomen corresponding to a position of the input on the input/output device;
d) counting a plurality of positions of fetal movements in said pregnant woman's abdomen over a recorded time period; and
e) saving the plurality of positions counted on a memory device.

10. The method of claim 9, wherein the schematic representation of the pregnant woman's abdomen displayed on the input/out device comprises: a vertical y-axis and a curved upwardly inflected x-axis generally perpendicular to the y-axis and crossing the y-axis at an origin representing a navel on the pregnant woman's abdomen, the axes thereby schematically dividing the pregnant women's abdomen into four quadrants.

11. The method of claim 9 wherein the at least one processor receives inputs from the input/output device representing the duration of fetal movements and records a plurality of durations of fetal movements corresponding with the positions of fetal movements over a recorded time period.

12. The method of claim 11 wherein at least one processor receives inputs from the input/output device representing an intensity of said fetal movement; and records and saves in the memory device a plurality of intensity ratings of fetal movements corresponding with the positions of fetal movements recorded over the recorded time period.

13. The method of claim 11 wherein information relating to the fetal movement over a recorded time period may be communicated by a means of wired or wireless communication or a combination thereof to or from a remote computing device.

14. The method of claim 9, comprising the additional steps:
f) presenting qualitative questions and associated input answer fields thereto, and
g) receiving input from the pregnant woman and associating and recording the input from said fields with information relating to a recorded time period.

15. The method of claim 9 wherein fetal movements are recorded during discrete time periods and are compared to fetal movements recorded in different discrete time periods.

16. The method of claim 9, wherein the at least one processor evaluates: differences between fetal movements recorded in the same time period or differences between fetal movements recorded in different time periods or a combination thereof.
